(19) United States
(12) Patent Application Publication
(10) Pub. No.: US 2025/0360114 A1
(43) Pub. Date: Nov. 27, 2025

(54) INTRAVENOUS RIFABUTIN FORMULATION AND METHODS OF MAKING

(71) Applicant: BIOVERSYS AG, Basel (CH)

(72) Inventors: Julian Schill, Basel (CH); Jürgen Jäger, Efringen-Kirchen (DE); Marc Gitzinger, Basel (CH); Glenn E. Dale, Basel (CH)

(21) Appl. No.: 19/181,189

(22) Filed: Apr. 16, 2025

Related U.S. Application Data
(60) Provisional application No. 63/635,136, filed on Apr. 17, 2024.

(51) Int. Cl. A61K 31/438; A61K 9/00; A61K 9/14

(52) U.S. Cl. CPC A61K 31/438; A61K 9/0019; A61K 9/14

(57) ABSTRACT

The invention provides compositions and methods of creating a rifabutin powder suitable for IV formulation preparation.

### FIELD OF THE INVENTION

The invention is generally related to a rifabutin powder with enhanced purity, suitable for preparing IV-formulations.

### BACKGROUND

Millions of people die each year from bacterial infections, and the numbers are increasing due to the spread of multi-drug resistant (MDR) and extensively-drug resistant (XDR) strains of bacteria. For example, according to official estimates, the annual number of deaths due to infections from antibiotic-resistant bacteria in the United States, European Union, and India alone is over 100,000, and some experts believe that official tallies are vast underestimates because the full impact of antibiotic resistance is still unknown.

Of particular concern, according to the World Health Organization's priority list, is Acinetobacter baumannii a gram-negative bacterium belonging to the so called ‘ESKAPE’ pathogens. ESKAPE pathogens, often resistant to multiple antimicrobials, are a major cause of pneumonia, meningitis, infections of the urinary tract, blood, or skin, and other deadly illnesses. They are often readily transmissible in hospital environments, and most infections are nosocomially acquired. As such, these pathogens present a significant risk to those with compromised immune systems, such as hospital patients and individuals suffering from HIV/AIDS.

ESKAPE pathogens are considered opportunistic and exhibit a high degree of genetic plasticity, which allows the quick development of antibiotic resistance. Once resistance is acquired or, once the patient becomes too sick, the only way to deliver sufficient antibiotic treatment is through intravenous (IV) administration. Unfortunately, some antibiotics do not pass the standards set out by the Food and Drug Administration (FDA), The European Medical Association (EMA), and other regulatory bodies, for use as an IV antibiotic, due to issues during development.

The FDA, EMA, and other regulatory bodies outline strict standards for manufacturers regarding drug quality and safety. Especially for parenteral products the quality requirements are very strict e.g. the number of sub-visible particles allowed in an IV-formulation according to the US and European pharmacopeia must be not more than 25 particles/mL greater 10 μm and not more than 3 particles/mL greater 25 μm for preparations with a nominal content of more than 100 mL, respectively not more than 6000 particles per container greater 10 μm and not more than 600 particles greater 25 μm for containers with a nominal content of less than 100 mL. Rifabutin is a spiro-piperidyl-rifamycin derived from rifamycin-S, also known as Mycobutin®, that was approved for oral formulation in 1992. Mycobutin® (150 mg capsules) capsules were indicated for the prevention of disseminated Mycobacterium avium complex (MAC) disease in patients with advanced HIV infection.

It was observed that all tested rifabutin, conforming to current USP and EP monographs and from different providers (“standard grade”), generates an excess of sub-visible particles when attempts are made to formulate it in certain aqueous solutions for intravenous administration, e.g. saline. This excess of sub-visible particles is caused by the presence of small amounts of a mix of impurities which are present in the standard grade rifabutin API. These impurities so far are unknown/not described in the current pharmacopeial methods and therefore are not controlled and qualified. Singularly they are below the reporting threshold for unknown impurities, however, sum up to about 0.3-0.5%, which was gravimetrically determined after filtration of standard grade rifabutin formulated in aqueous media suitable for intravenous injection. Thus, there is a need in the art to produce rifabutin powder with increased purity that can be formulated in aqueous media suitable for intravenous injections.

### SUMMARY

The present invention provides improved rifabutin powder of a higher purity that is suitable for formulation into a drug product for intravenous administration (“IV-grade”). The invention provides IV-grade rifabutin that, when used to prepare IV-formulations in aqueous media suitable for intravenous injection, the sub-visible particle counts are well below the accepted limit of the US and European pharmacopeia for IV drug products, which is not more than 25 particles/mL greater 10 μm and not more than 3 particles/mL greater 25 μm for preparations with a nominal content of more than 100 mL, respectively not more than 6000 particles per container greater 10 μm and not more than 600 particles greater 25 μm for containers with a nominal content of less than 100 mL.

In one aspect, the invention comprises separating impurities in standard grade rifabutin by selectively dissolving the rifabutin in an alkane solution via extraction processes as described in the method section. In a preferred embodiment, rifabutin API is dissolved in an alkane solution in which sub-visible particles forming impurities are not soluble and remain undissolved. The alkane solution is then distilled away from the rifabutin solution, resulting in a rifabutin powder that is significantly purer and is suitable for IV administration. Additionally, to the removal of many unknown impurities, also some of the known impurities such as rifabutin N-oxide are significantly reduced during the purification process. The resulting IV-grade rifabutin powder is then formulated in an aqueous solution suitable for intravenous administration.

Methods and compositions of the invention comprise dissolving rifabutin in certain alkanes. In certain embodiments, the invention includes a composition in which the solvent is n-pentane, n-hexane, n-heptane, or cyclohexane.

As a result of the purification processes of the invention, it is possible to generate IV-grade rifabutin, which upon formulation in aqueous media suitable for IV administration contains practically no sub-visible particles forming impurities. Accordingly, purification processes of the invention result in the production of IV-grade rifabutin formulations of higher purity and in which there are substantially no sub-visible particles. The present invention encompasses any method in which rifabutin powder is solubilized in an alkane solvent. The present invention also encompasses any method wherein, once combined, the mixture is exposed to a solid-liquid extraction process and rifabutin suitable for IV administration is extracted, which forms substantially no sub-visible particles in aqueous formulation. The mixture can be heated to temperatures up to a maximum of 50° C. during the extraction process to avoid degradation of rifabutin.

According to another embodiment of the present invention, the solid-liquid extraction process is conducted in one or more reaction vessels connected by one or more tubular connections. The reaction vessels and tubular connections may further contain one or more filters to retain any sub-visible particles forming impurities and one or more cooling vessels, thus producing rifabutin powder that can be formulated into an intravenous solution.

In another embodiment, an adsorbent, e.g. silica gel, zeolite or activated alumina, is used to trap rifabutin present in the alkane solvent during the extraction process. Rifabutin trapped on the adsorbent can be eluted by a solvent with higher elution strength, e.g. acetone, to obtain rifabutin that can be formulated into an intravenous solution.

According to another embodiment, the rifabutin is mixed with a solvent at a temperature of at most about 50° C. to completely dissolve rifabutin while keeping sub-visible particle forming impurities undissolved and avoid degradation of rifabutin. The mixture is then filtered to remove undissolved impurities and the solvent is evaporated, resulting in IV-grade rifabutin powder.

### BRIEF DESCRIPTION OF THE DRAWINGS

FIG. 1 shows the solubility of isolated impurities in multiple solvents.

FIG. 2 shows the solubility of isolated impurities in multiple solvents.

FIG. 3 illustrates purification method 1.

FIG. 4 shows the binding of rifabutin to different silica gels.

FIG. 5 shows the binding of rifabutin to silica gel in the presence of different solvents.

FIG. 6 illustrates small scale purification using method 2.

FIG. 7 illustrates large scale purification using method 2.

### DETAILED DESCRIPTION

The present disclosure provides compositions and methods for generating IV-grade rifabutin. Rifabutin powder resulting from the extraction methods provided herein is free of impurities which would lead to sub-visible particles in certain aqueous formulations. The rifabutin powder that is obtained through practice of the invention can be formulated as an IV preparation. In particular, methods of the invention comprise solubilizing standard grade rifabutin API in an alkane solution. The alkane solution is used to dissolve rifabutin during the manufacturing process, leaving impurities that would lead to sub-visible particle formation in certain aqueous formulations, undissolved in the alkane. Rifabutin has a low solubility in alkanes. Thus, in a preferred embodiment, extraction of rifabutin is conducted via a solid-liquid extraction process that results in solubilization of small amounts of rifabutin at a time. The alkane is distilled away from the rifabutin solution, resulting in a purified rifabutin powder. The impurities that would lead to sub-visible particles remain undissolved in the solubilization process and are filtered off.

The invention contemplates a composition of IV-grade rifabutin comprising purified rifabutin powder formulated in an aqueous solution for IV administration.

### IV-grade Rifabutin

Rifabutin, conforming to current USP and EP monographs, generates an excess of sub-visible particles when attempts are made to formulate it in certain aqueous solutions for intravenous administration, e.g. saline. Due to this particle formation, such formulations not only exceed the limits set by the US and European pharmacopeias for sub-visible particles in parenteral formulations but also bear the risk of interrupting the administration of the drug due to blocking of the filters in the infusion line, ultimately resulting in treatment failure.

As these particle forming impurities are not described in the current pharmacopeial analytical methods, a new testing method was developed which is described in the methods below. In brief, rifabutin powder is dissolved in a mixture of dimethyl isosorbide, acetic acid and water. Subsequently, the obtained solution is diluted in 0.9% saline to a final concentration of 10 mg/mL and sub-visible particles are determined. The established limits for this test are: (1) not more than 12.5 particles/mL greater than 10 μm in diameter, and (2) not more than 3 particles/mL greater than 25 μm in diameter. Considering that current concentrations used for the intravenous administration of rifabutin are well below 10 mg/mL, these limits are much more stringent than the current pharmacopeial limits.

### Generation of Purified Rifabutin Powder

Methods for the preparation of purified rifabutin powder that can then be formulated in aqueous solution preferably include a mixture comprising an alkane solvent and standard grade rifabutin. Some embodiments of the present invention may also comprise a class 3 solvent or a solvent selected from a group consisting of: n-pentane, n-hexane, n-heptane, or cyclohexane, combined with rifabutin. Solvents of the present invention are chosen for their ability to dissolve rifabutin while not dissolving impurities which would form sub-visible particles in certain aqueous formulations. It is important that these impurities remain fully insoluble in the chosen solvent. A solvent screening was performed in order to find a solvent that selectively dissolves rifabutin, while keeping impurities undissolved. For example, solvents such as acetone, ethanol, anisole, ethyl formate, and others are not ideal, as these impurities are either soluble or slightly soluble in those solvents.

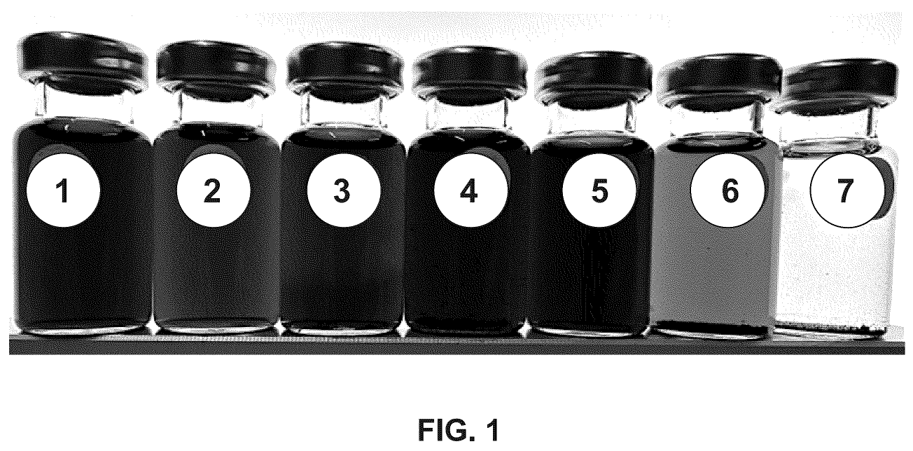

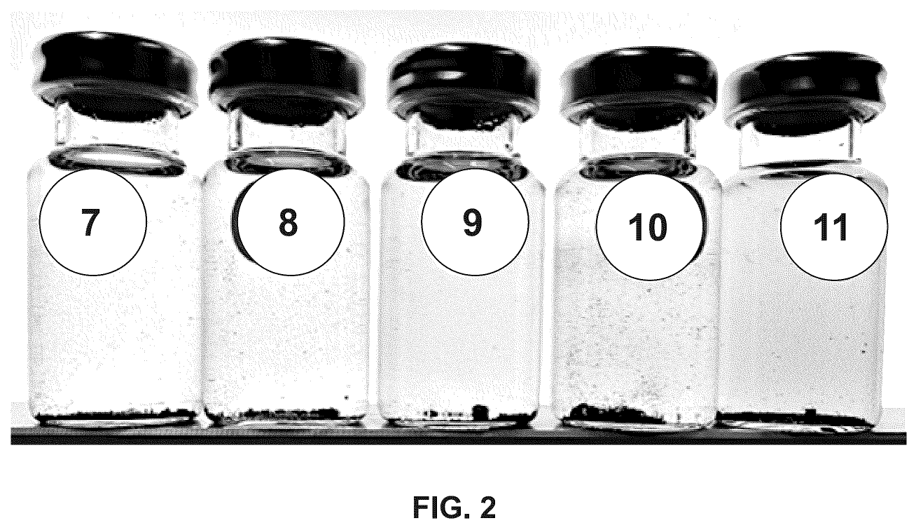

FIGS. 1 and 2 show the solubility of sub-visible particles forming impurities, isolated from an aqueous solution prepared from standard grade rifabutin powder, in various solvents. Here, these isolated impurities were mixed with a selection of different solvents in order to test their solubility. A darker color indicates more dissolved impurities in solution.

As can be seen in vials 1-5, sub-visible particles forming impurities from the standard grade rifabutin powder were highly soluble in acetone, ethanol, tert-butanol, anisole, and ethyl formate. This indicates that these are poor candidates for obtaining IV-grade rifabutin, as both the rifabutin and sub-visible particles forming impurities would be dissolved.

Vial 6 shows that sub-visible particle forming impurities from the standard grade rifabutin powder were only slightly soluble in tert-butyl methyl ether (MTBE), as Vial 6 exhibits a slightly lighter color than vials 1-5. Vial 6 also exhibits more undissolved impurities at the bottom of the vial than vials 1-5. Vial 11 shows that the isolated impurities were only marginally soluble in diisopropyl ether, as can be seen by the only slightly colored solution and large amount of undissolved impurities.

Vials 7-10 show that with the tested alkane solvents (n-pentane, n-hexane, cyclohexane and n-heptane) the solution stays colorless, and the impurities remain undissolved. This makes the alkane solvents strong candidates for the use in the rifabutin purification process.

Methods of the present invention avoid degradation of the rifabutin itself throughout the extraction processes. As such, it is a preferred embodiment of the present invention to perform the purification at temperatures at or below 50° C. and in solvents in which the impurities leading to sub-visible particles remain insoluble.

### Methods for Producing Purified Rifabutin

### Method 1

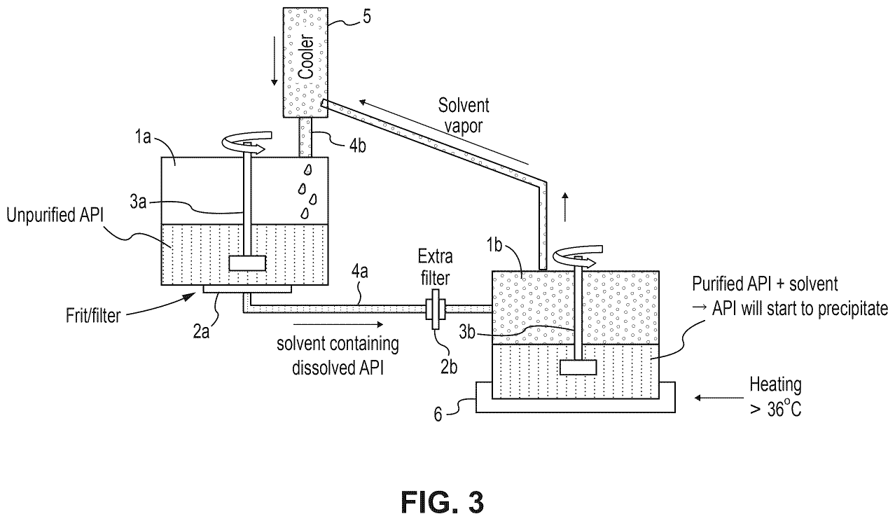

FIG. 3 shows an exemplary method for the extraction of IV-grade rifabutin from a rifabutin solvent mixture. Rifabutin powder and a solvent are combined in vessel (1a) and agitated with mixing element (3a) to form a mixture. The mixture passes through filter (2a) which retains undissolved rifabutin and particles that may be contained in the mixture. After passing through filter (2a) the filtered mixture enters tubular connection (4a). The filtered mixture travels through tubular connection (4a) and passes through optional filter (2b). Once the filtered mixture passes through filter (2b) it enters vessel (1b) where the filtered mixture is heated by heating element (6) and agitated by mixing element (3b). As soon as the concentration of rifabutin exceeds its solubility due to evaporation of the solvent, IV-grade rifabutin precipitates from the filtered mixture. The evaporated solvent is cooled down (5) and the condensate is transferred through tubular connection (4b) back into vessel (1a). This process runs continuously until the purification is complete and only undissolved impurities remain in vessel (1a). After cooling down the content of vessel (1b) the precipitated rifabutin is filtered off and dried. The purified powder obtained is then formulated in an aqueous saline solution for intravenous administration without the formation of extended numbers of sub-visible particles.

Methods disclosed herein are not intended to limit the methods of making IV-grade rifabutin in any way. The formulation of IV-grade rifabutin powder according to the instant methods can be completed via any process of solid-liquid extraction. The methods disclosed herein are intended to be used as an exemplary method of making an IV-grade rifabutin.

### Example 1

5 g of rifabutin were mixed with 85 g of sand and filled into a cellulose extraction thimble (33×94 mm). The filled extraction thimble was placed in a Soxhlet extractor and the whole system assembled. Approx. 160 mL of pentane were filled into the system and the water bath was heated to 50° C. to start the extraction. Extraction was run for a total duration of 7.5 hours. During the extraction procedure, rifabutin is dissolved and collected in the bottom flask of the Soxhlet extractor, where it precipitates, while undissolved impurities are retained in the extraction thimble. Once completed, the collected rifabutin was dried under vacuum to remove residual solvent. The process yield was >96%

HPLC analysis shows an overall increase in purity of about 1% after purification (Table 1). As shown by the results from the sub-visible particle test (Table 2), sub-visible particle forming impurities are in the acceptable range.

TABLE 1
HPLC results, Method 1, Example 1

% Area

| Peak | RRT | original | purified | comment |
|---|---|---|---|---|
| 1 | 0.37 | 0.06 | 0.06 | |
| 2 | 0.46 | 0.06 | — | 16-desacetylrifabutin |
| 3 | 0.55 | 0.08 | 0.07 | Hydroxy rifabutin |
| 4 | 0.74 | 0.09 | — | |
| 5 | 0.78 | 0.07 | — | |
| 6 | 0.82 | 0.06 | 0.05 | |
| 7 | 0.85 | 0.05 | — | |
| 8 | 0.91 | 0.23 | 0.23 | |
| 9 | 0.94 | 0.27 | 0.19 | |
| 10 | 0.98 | 0.15 | 0.16 | Rifabutin 14 R epimer |
| 11 | 1.00 | 97.54 | 98.1 | Rifabutin |
| 12 | 1.20 | 0.29 | 0.09 | Rifabutin N-oxide |
| 13 | 1.26 | 0.05 | 0.06 | |
| 14 | 1.53 | 0.22 | 0.24 | Didehydrorifabutin |
| 15 | 1.61 | 0.08 | — | |
| 16 | 1.86 | 0.05 | — | |
| 17 | 2.42 | 0.1 | 0.1 | |

TABLE 2
Sub-visible particles test results obtained
with purified drug substance (Method 1)

particles/mL

| Particle size | original | purified |
|---|---|---|
| ≥10 μm | 138.3 | 8.4 |
| ≥25 μm | 16.3 | 0.4 |

### Further Examples

Multiple different rifabutin batches from different suppliers were purified according to the procedure described in Example 1. As shown in Table 3 all purified batches were clearly within the set limit of not more than 12.5 particles/mL greater 10 μm and not more than 1.5 particles/mL greater 25 μm.

TABLE 3
Sub-visible particles test results for different purified
and unpurified rifabutin batches, particles/mL

Manufacturer of standard grade rifabutin

| | A | A | A | A | B | B |
|---|---|---|---|---|---|---|
| batch | 1 | 1a | 2 | 2a | 3 | 3a |
| grade | standard | IV | standard | IV | standard | IV |
| ≥10 μm | 276.1 | 1.7 | 1804.0 | 7.6 | 438.1 | 5.3 |
| ≥25 μm | 29.7 | 0.1 | 98.7 | 0.2 | 23.3 | 0.6 |

### Method 2

During different trials trying to find alternatives for the sand used in example 1, it was observed that rifabutin strongly adsorbs to silica gel and is not eluted by alkane solvents. One limitation of the previously described purification method (Method 1) is the separation of rifabutin and solvent by distillation which is rather slow and requires continuous heating of the purified rifabutin. In this second method distillation is replaced by introducing a column filled with silica gel, which efficiently binds the rifabutin and separates it from the alkane solvent. While rifabutin remains bound to the silica in the presence of alkane solvent, it can be easily eluted with other solvents, such as acetone.

### Silica Selection

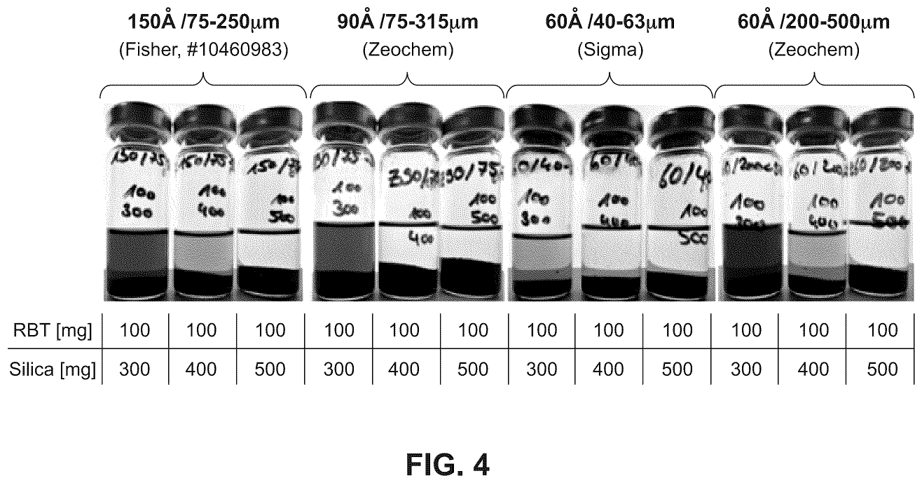

Multiple silica gels were tested to select the most suitable diameter and pore size. As shown in FIG. 4, 100 mg of rifabutin and different amounts and types of silica were mixed in a glass vial and 4 mL of pentane were added. Vials were sealed using stoppers and crimp caps and put on a roller mixer until no further changes in solution color were observed. The best results were obtained with silica gel having a pore size of 90 Å and a diameter of 75-315 μm binding rifabutin in a 1:4 ratio (400 mg rifabutin per 100 mg of silica gel). Although the binding capacity of silica gel with 60 Å and smaller diameter is slightly higher, it was deemed less suitable as binding is much slower which might increase the processing time.

### Solvent Selection

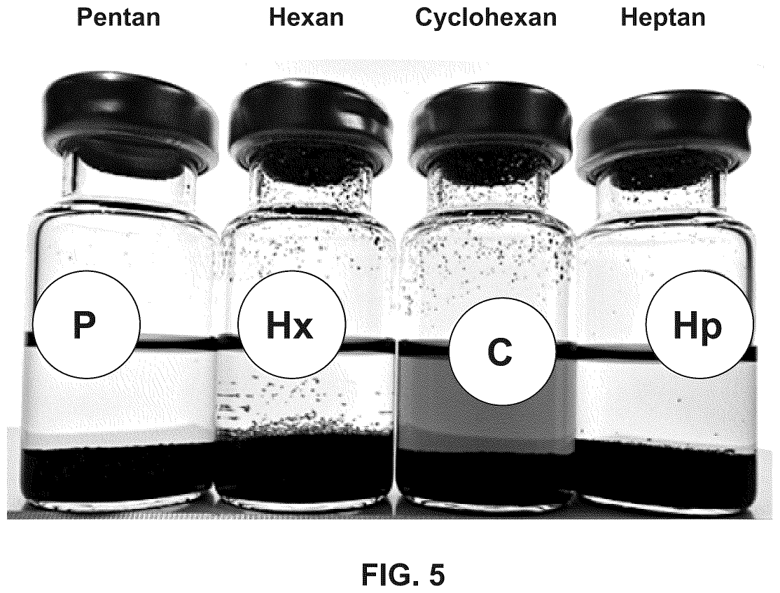

Using the same silica and ratio determined to be most suitable in the previous experiment (90 Å pore size, 75-315 μm diameter and a rifabutin to silica ratio of 1:4) binding of rifabutin was also tested with other alkane solvents. As shown in FIG. 5, binding with n-hexane and n-heptane worked similar to n-pentane, while cyclohexane is less suitable as rifabutin does not completely bind to the silica.

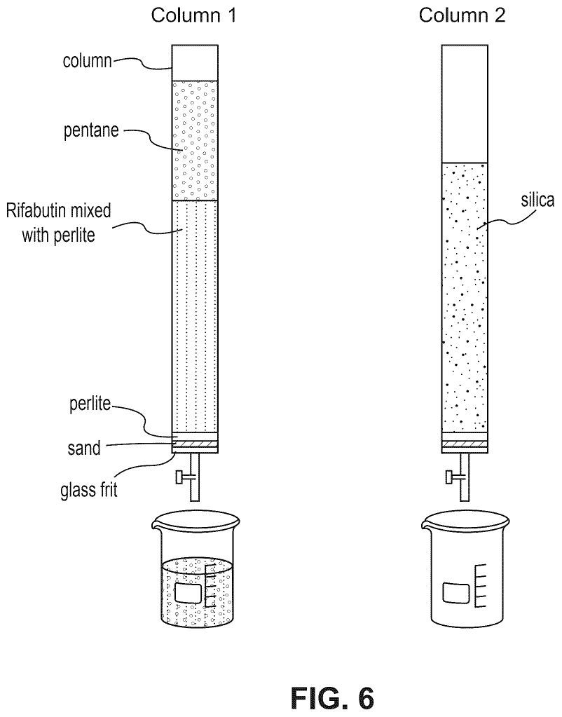

### Method 2 Lab Scale (FIG. 6)

Rifabutin is mixed with perlite in a 1:2 ratio and packed into a glass column. A second column is packed with silica gel, using a silica to rifabutin ratio of about 1:5. Both columns contain a glass frit onto which a layer of sand and perlite is added prior to packing, in order to retain the undissolved rifabutin or silica gel respectively. An alkane solvent is added to column 1. While running through the column, the alkane solvent dissolves the rifabutin. The alkane solvent containing rifabutin is collected and transferred to the second column. While the solution runs through column 2 rifabutin adsorbs to the silica. The alkane solvent depleted of rifabutin is subsequently added back to column 1. These steps are repeated until all rifabutin is adsorbed to the silica in column 2 and only insoluble impurities remain in column 1. Rifabutin is then eluted from column 2 using acetone. In a last step the solvent is evaporated to obtain purified (IV-grade) rifabutin.

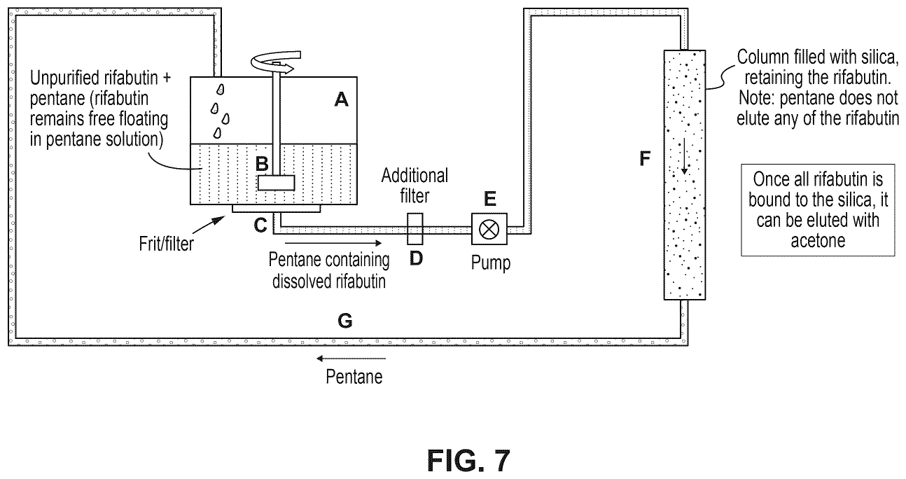

### Method 2 Large Scale (FIG. 7)

Rifabutin and an alkane solvent are added into a reactor (A) and mixed with a mechanical stirrer (B) to dissolve the rifabutin. The solution flows through a filter in the bottom of the reactor (C), retaining undissolved rifabutin, and an additional filter D. Using a pump (E) the solution is pumped through a column (F) filled with silica gel. Rifabutin is adsorbed on the silica gel in the column. The alkane solvent depleted of rifabutin flows through tubing (G) back to the reactor (A). The alkane solvent is circulated through the system until all rifabutin is adsorbed to the silica gel in the column (F) and only insoluble impurities suspended in the alkane solvent remain in the reactor (A). After the extraction phase, the rifabutin is eluted from the column (F) using acetone. In a last step the solvent is evaporated at reduced pressure to obtain purified (IV-grade) rifabutin.

### Example 2

10 g of rifabutin and 20 g of perlite were mixed and filled into a glass column (column 1). A Second column was packed with 55 g of silica gel (90 Å, 75-315 μm). The bottom of the glass columns contained a glass frit onto which sand and additional perlite was added to retain the undissolved rifabutin or silica gel respectively.

Pentane was ran through column 1 to dissolve rifabutin and collected in a beaker. After approx. 200 mL of pentane were collected the rifabutin in pentane solution was ran through column 2. Rifbautin is adsorbed on the silica gel and the almost colorless pentane was collected in a beaker before then again being transferred to column 1 to dissolve additional rifabutin. These steps were repeated about 20 times, until all rifbutin was transferred from column 1 to column 2 where it was adsorbed to the silica gel and only insoluble impurities remained in column 1.

Rifabutin was then eluted from column 2 using approximately 150 mL of acetone. The solvent was evaporated under vacuum to obtain purified (IV-grade) rifabutin with a yield of 97.5%.

HPLC analysis shows an overall increase in purity of about 2% after purification (Table 4). As shown by the results from the sub-visible particle test (Table 5), sub-visible particle forming impurities are in the acceptable range.

TABLE 4
HPLC results, Method 2, Example 2

% Area

| Peak | RRT | original | purified | Impurity |
|---|---|---|---|---|
| 1 | 0.47 | 0.1 | — | 16-desacetylrifabutin |
| 2 | 0.49 | 0.1 | 0.08 | |
| 3 | 0.55 | 0.16 | 0.15 | Hydroxy rifabutin |
| 4 | 0.62 | 0.05 | — | |
| 5 | 0.72 | 0.06 | — | |
| 6 | 0.78 | 0.12 | — | |
| 7 | 0.84 | 0.12 | 0.06 | |
| 8 | 0.89 | 0.13 | 0.07 | |
| 9 | 0.92 | 0.44 | 0.39 | Rifabutin 14 R epimer |
| 10 | 1.00 | 96.43 | 98.49 | RBT |
| 11 | 1.19 | 0.45 | — | Rifabutin N-oxide |
| 12 | 1.58 | 0.22 | 0.2 | Didehydrorifabutin |
| 13 | 1.70 | 0.09 | 0.09 | |
| 14 | 1.89 | 0.1 | — | |
| 15 | 1.98 | 0.11 | — | |
| 16 | 2.00 | 0.12 | — | |
| 17 | 2.03 | 0.06 | — | |
| 18 | 2.06 | 0.14 | — | |
| 19 | 2.08 | 0.06 | — | |
| 20 | 2.10 | 0.1 | — | |

TABLE 5
Sub-visible particles test results obtained
with purified drug substance (Method 2)

particles/mL

| Particle size | original | purified |
|---|---|---|
| ≥10 μm | 1804.0 | 2.6 |
| ≥25 μm | 98.7 | 0.0 |

### Method 3

Besides using continuous extraction processes, as described in method 1 and method 2, purification can also be achieved by simply dissolving standard grade rifabutin in a sufficient amount of solvent and remove the undissolved impurities by filtration.

Rifabutin is mixed with one of the alkane solvents in a ratio that allows complete dissolution of rifabutin. The solution is stirred until rifabutin is fully dissolved and subsequently filtered. After filtration the solvent is evaporated to obtain purified (IV-grade) rifabutin powder.

### Example 3

1.5 g rifabutin were mixed with 250 mL of n-heptane and heated at 50° C. under stirring for 1 hour. The solution was filtered through perlite to remove any undissolved impurities. The heptane was removed using a rotary evaporator to obtain purified (IV-grade) rifabutin powder. The yield was 95.3%.

HPLC analysis shows an overall increase in purity of about 2% after purification (Table 6). As shown by the results from the sub-visible particle test (Table 7), sub-visible particle forming impurities are in the acceptable range.

TABLE 6
HPLC results, Method 3, Example 3

% Area

| Peak | RRT | original | purified | Impurity |
|---|---|---|---|---|
| 1 | 0.32 | 0.06 | — | |
| 2 | 0.46 | 0.1 | — | 16-desacetylrifabutin |
| 3 | 0.49 | 0.11 | 0.09 | |
| 4 | 0.55 | 0.18 | 0.15 | Hydroxy rifabutin |
| 5 | 0.62 | 0.07 | — | |
| 6 | 0.68 | 0.07 | — | |
| 7 | 0.72 | 0.07 | — | |
| 8 | 0.78 | 0.16 | 0.08 | |
| 9 | 0.84 | 0.1 | 0.08 | |
| 10 | 0.88 | 0.14 | 0.11 | |
| 11 | 0.93 | 0.46 | 0.44 | Rifabutin 14 R epimer |
| 12 | 1.00 | 95.94 | 98.02 | RBT |
| 13 | 1.19 | 0.52 | 0.12 | Rifabutin N-oxide |
| 14 | 1.49 | 0.25 | 0.22 | |
| 15 | 1.55 | 0.13 | 0.10 | Didehydrorifabutin |
| 16 | 1.57 | 0.05 | — | |
| 17 | 1.66 | 0.14 | — | |
| 18 | 1.74 | 0.11 | — | |
| 19 | 1.75 | 0.14 | — | |
| 20 | 1.78 | 0.08 | — | |
| 21 | 1.81 | 0.14 | 0.07 | |
| 22 | 1.82 | 0.07 | — | |
| 23 | 1.84 | 0.1 | — | |

TABLE 7
Sub-visible particles test results obtained
with purified drug substance (Method 3)

particles/mL

| Particle size | original | purified |
|---|---|---|
| ≥10 μm | 1804.0 | 7.1 |
| ≥25 μm | 98.7 | 0.2 |

### Testing Methods

### HPLC Analysis

Weigh approx. 10 mg of rifabutin into a clean glass vial. Dissolve in 1 ml acetonitrile and dilute to 1 mg/mL using diluent.
Chromatographic system: HPLC Waters Alliance equipped with UV detector or equivalent
Software: Empower 3 System of equivalent
Column: Intersil ODS-3V 250×4.6 mm 5 μm
Flow: 1.0 mL/min
Diluent: 0.1% Triethylamine/Acetonitrile 8/2 pH 6.0 with trifluoracetic acid (TFA)
Column temperature: 50° C.
Sample concentration: 1 mg/mL
Dissolution phase: Acetonitrile/diluent 1/9
Injection volume: 20 μl
Detector UV: 254 nm
Analysis time: 90 min
Acquisition time: 90 min
Mobile phase: Phase A: 0.1% Triethylamine/Acetonitrile 8/2 pH 2.5 with TFA Phase B: 0.1% Triethylamine/Acetonitrile 1/9 pH 2.5 with TFA
Gradient:

| Time | Phase | Phase |
|---|---|---|
| [min] | A [%] | B [ % ] |
| 0 | 75 | 25 |
| 35 | 75 | 25 |
| 45 | 45 | 55 |
| 50 | 40 | 60 |
| 60 | 30 | 70 |
| 70 | 30 | 70 |
| 71 | 0 | 100 |
| 80 | 0 | 100 |
| 81 | 75 | 25 |
| 91 | 75 | 25 |

As particle forming impurities are not detected by current pharmacopeial analytical methods, a new method was developed to test the drug substance regarding its suitability for the preparation of intravenous formulations.

### Sub-visible Particle Test

About 400 mg of rifabutin is dissolved in 2 mL of a mixture of isosorbide dimethyl ether (DMI) and acetic acid in water (40% DMI, 2.4% acetic acid). The obtained solution is filtered through a 0.2 μm Nylon filter and 1.5 mL are added to 23.5 mL of 0.9% saline solution to a final concentration of 10 mg/mL. Solutions are stirred for 15 min on a roller mixer to allow sub-visible particle formation.

Sub-visible particles are determined using a suitable instrument (e.g. PAMAS SVSS particle counter). Particle numbers in the 10 mg/mL solution should be not more than 12.5 particles/mL greater 10 μm and not more than 1.5 particles/mL greater 25 μm to pass the test.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A purified rifabutin powder from which sub-visible particles have been removed.

2. The rifabutin powder of claim 1, wherein a 10 mg/mL aqueous solution comprising the rifabutin powder has no more than about 12.5 particles/mL that are greater 10 μm in diameter and no more than about 1.5 particles/mL greater than 25 μm in diameter.

3. The rifabutin powder of claim 2, wherein the aqueous solution is suitable for intravenous administration.

4. The rifabutin powder of claim 1, wherein the sub-visible particles forming impurities are insoluble in an alkane solvent.

5. The rifabutin powder of claim 4, wherein the alkane solvent is selected from the group consisting of n-pentane, n-hexane, n-heptane, and cyclohexane.

6. The alkane solvent of claim 5, wherein the alkane solvent is a class 3 solvent.

7. A pharmaceutical composition comprising the rifabutin powder of any one of claims 1-6, and a pharmaceutically acceptable solvent and/or excipient.

8. The pharmaceutical composition of claim 7, wherein the pharmaceutical composition is suitable for intravenous administration.

9. A method of producing a rifabutin for intravenous administration, the method comprising: combining a rifabutin powder with an alkane solvent to create a mixture;
exposing the mixture to a solid-liquid extraction process; and extracting purified rifabutin from the mixture.

10. The method of claim 9, wherein the extracting step comprises distilling the solvent away from the rifabutin powder.

11. The method of claim 9, wherein the extracting step comprises trapping the rifabutin using a silica gel.

12. The method of claim 9, wherein the solid-liquid extraction process is repeated.

13. The method of claim 9, wherein an amount of rifabutin is completely dissolved in an alkane solvent.

14. The method of claim 9, wherein insoluble impurities are removed with one or more filters.

15. The method of claim 9, wherein the mixture is heated to or above about 20° C.

16. The method of claim 9, wherein the mixture is heated at or lower than about 50° C.

17. The method of claim 9, wherein the method is conducted in one or more reaction vessels that are connected by one or more connections.

18. The method of claim 9, wherein the alkane solvent is selected from the group consisting of n-pentane, n-hexane, n-heptane, and cyclohexane.

19. The method of claim 18, wherein the alkane solvent is a class 3 solvent.
